(19) United States
(12) Patent Application Publication
(10) Pub. No.: US 2026/0198771 A1
(43) Pub. Date: Jul. 16, 2026

(54) Method and Device for Determining Thickness of a Post-Lens Tear Film

(71) Applicant: TELEFONAKTIEBOLAGET LM ERICSSON (PUBL), Stockholm (SE)

(72) Inventors: Peter Ökvist, Luleå (SE); Tommy Arngren, Sõdra Sunderbyn (SE); Andreas Kristensson, Södra Sandby (SE); Niklas Lindskog, Lund (SE)

(21) Appl. No.: 19/132,256

(22) PCT Filed: Dec. 22, 2022
(86) PCT No.: PCT/SE2022/051230

§ 371 Date: May 22, 2025

(51) Int. Cl. A61B 3/10; G01B 11/06; G02C 7/04; G02C 11/00

(52) U.S. Cl. CPC A61B 3/101; G01B 11/06; G02C 7/04; G02C 11/10

(57) ABSTRACT

A method is disclosed for determining thickness of a post-lens tear film as function of time. The method is performed in a device placed on a smart contact lens placed on an eye. The method comprises performing measurements of light intensity of light transmitted from a light emitter and received by a light detector. The light emitter and light detector are both arranged on the smart contact lens. The method comprises determining the thickness of the post-lens tear film based on the performed measurements, on wavelength of the emitted light and on refraction index of tear fluid between the smart contact lens and the cornea of the eye.

### TECHNICAL FIELD

The technology disclosed herein relates generally to the field of smart contact lenses, and in particular to devices and methods for determining thickness of a post-lens tear film as function of time.

### BACKGROUND

Contact lenses are thin, clear discs worn in eyes to improve vision of a user. The contact lenses float on a tear film that covers the cornea of the eye. A tear exchange beneath the contact lens facilitates ongoing fluid replenishment in the eye, between an ocular surface and the lens. Such exchange is considerably lower during the wear of soft lenses compared to wear of rigid lenses. As a result of reduced tear exchange, the accumulation of tear film debris and metabolic by-products between a cornea and a soft contact lens increase, which may lead to complications.

Traditionally, the tear exchange has been described as having a leading role in delivering oxygenated tears to the cornea behind non-permeable, polymethyl-methacrylate (PMMA) contact lens materials, but modern, highly oxygen transmissible silicone hydrogel (SiHy) lenses have virtually eliminated hypoxic complications, and the significance of tear exchange has been redefined. It is still considered essential that tear exchange occurs in an attempt to reduce post-lens debris, such as metabolic byproducts that stagnate between the lens and cornea. This desired tear exchange is particularly important in extended and continuous wear, which is today understood to plausible contribute to the onset of adverse events, by altering the epithelial barrier function.

Various methods for measuring thickness of pre- and post-contact lens tear films are known. In common for these methods is their time-consuming approach for assessment and measurements of pre- and post-lens tear film thicknesses. The methods typically involve measuring by using more or less advanced equipment such as imaging spectrograph and optical pachymetry devices. The measurement arrangements and setups are large, and patients have to go to an ophthalmologist or an optician for the assessment of tear film thickness.

In view of the above noted difficulties, it is clear that there is a need for improvements in view of contact lenses and the health of their users' eyes and eyesight.

### SUMMARY

An objective of embodiments herein is to address and improve various aspects relating to contact lenses. A particular objective is to provide a smart contact lens enabled to establish whether there is sufficient tear fluid exchange in an eye. Another objective is to provide a method for facilitating in-vivo assessments of a post-contact tear film thickness. Still another objective is to increase the time periods that the contact lenses can be worn. These objectives and others are achieved by the methods, devices, computer programs and computer program products according to the appended independent claims, and by the embodiments according to the dependent claims.

These objective and others are accomplished by a method and devices for determining thickness of the post-lens tear film.

According to a first aspect there is presented a method for determining thickness of a post-lens tear film as function of time. The method is performed in a device placed on a smart contact lens, which in turn is placed on an eye. The method comprises performing measurements of light intensity of light transmitted from a light emitter. The light is received by a light detector, wherein the light emitter and the light detector are both arranged on the smart contact lens. The method comprises determining the thickness of the post-lens tear film based on the performed measurements, on wavelength of the emitted light and on refraction index of tear fluid between the smart contact lens and the cornea of the eye.

According to a second aspect there is presented a computer program for determining thickness of a post-lens tear film as function of time. The computer program comprises computer code which, when run on processing circuitry of a device, causes the device to perform a method according to the first aspect.

According to a third aspect there is presented a computer program product comprising a computer program as above, and a computer readable storage medium on which the computer program is stored.

According to a fourth aspect there is presented a device for determining thickness of a post-lens tear film as function of time. The device may be placed on a smart contact lens in turn placed on an eye. The device is configured to perform measurements of light intensity of light transmitted from a light emitter and received by a light detector. The light emitter and light detector are both arranged on the smart contact lens. The device is configured to determine the thickness of the post-lens tear film based on the performed measurements, on wavelength of the emitted light and on refraction index of tear fluid between the smart contact lens and the cornea of the eye.

Advantageously, these aspects enable determining of thickness of post-contact lens tear film thickness and may be deployed on a smart contact lens. These aspects also enable providing the measurement data in real time during all day of use. These measurements may be sent to a user device and/or a central server for further analysis. Further, assessment data may be associated with a user's everyday context and the smart contact lens may be worn during longer time periods during a day.

Other objectives, features and advantages of the enclosed embodiments will be apparent from the following detailed disclosure, from the attached dependent claims as well as from the drawings.

Generally, all terms used in the claims are to be interpreted according to their ordinary meaning in the technical field, unless explicitly defined otherwise herein. All references to “a/an/the element, apparatus, component, means, module, action, etc.” are to be interpreted openly as referring to at least one instance of the element, apparatus, component, means, module, action, etc., unless explicitly stated otherwise. The actions of any method disclosed herein do not have to be performed in the exact order disclosed, unless explicitly stated.

### BRIEF DESCRIPTION OF THE DRAWINGS

The inventive concept is now described, by way of example, with reference to the accompanying drawings, in which:

FIGS. 1a and 1b are schematic drawings illustrating devices according to embodiments.

FIGS. 2a and 2b illustrate optical path length difference for light.

FIG. 3 illustrates a monochromatic interferometer contraption.

FIG. 4 is a flowchart of various embodiments of a method.

FIG. 5 is a schematic diagram showing functional units of a device according to an embodiment.

FIG. 6 is a schematic diagram showing functional modules of a device according to an embodiment.

FIG. 7 shows one example of a computer program product comprising computer readable means according to an embodiment.

### DETAILED DESCRIPTION

The inventive concept will now be described more fully hereinafter with reference to the accompanying drawings, in which certain embodiments of the inventive concept are shown. This inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description. Any action or feature illustrated by dashed lines should be regarded as optional.

Briefly, instead of using indirect measuring as in prior art, the present teachings enable a direct assessment of post-contact lens tear film thickness. Further, in contrast to the existing measurement arrangements and setups, the herein described method and devices for determining thickness of post-contact lens tear film thickness may be deployed on a contact lens and may provide measurement data in vivo during the user's daily use. The measurement data may also be sent to a user device and/or a central server for further analysis.

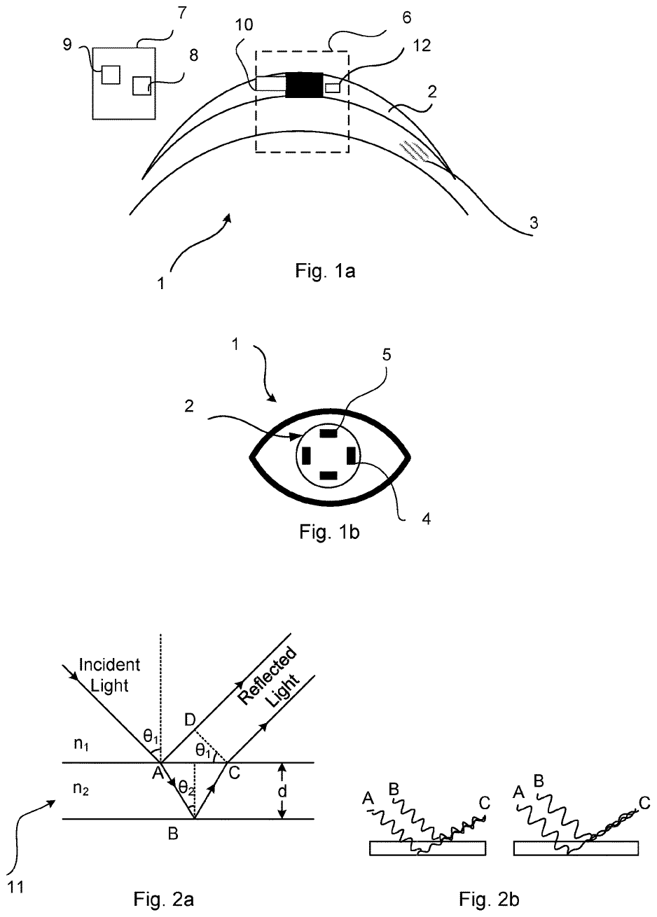

FIGS. 1a and 1b are schematic drawings illustrating, in a side view and view from above, respectively, a device according to embodiments. A smart contact lens 2 is provided, which is inserted on an eye 1. There is a layer of tear fluid between the eye 1 and the smart contact lens 2, which layer is denoted post-lens tear film 3. A device 6 is arranged on, or is an integrated part of, the smart contact lens 2 for determining thickness of the post-lens tear film 3 as function of time. The smart contact lens 2 thus comprises the device 6. The smart contact lens 2 (the device 6 thereof) may comprise a managing function instructing activation of light emitter and associated light detector, with a light transmission and sampling periodicity shorter than post-contact lens tear film thickness dynamics (i.e., sub-second) determines a first and subsequent interference maximums (or minimum depending on setup preference) at the light detector, for which a gap-distance measure is derived as function of light wavelength and material refraction indices. A managing device 7, external to the smart contact lens 2, is also provided.

An accumulated gap-distance change in correspondence to detected interference maximums and communicates said accumulated gap-distance as a function of time to a separate managing device such as smartwatch, smartwatch, or similar.

The device 6 of the smart contact lens 2 comprises means for determining thickness of a post-lens tear film as function of time. Such means may, for instance, comprise one or more light emitting components 4, such as e.g., Light Emitting Diode(s) (LED(s)) or μLED. The device 6 may further comprise one or more sensors 5 (or detectors), which may, for instance, be photosensors, point sensors, or linear array sensors. An optical element to guide light from a sensing area to the sensor(s) 5 is preferably also part of the device 6. Any optical light guiding element able to direct light from the source (light emitter) to the sensor(s) 5 may be used.

The device 6, and thus the smart contact lens 2, may optionally, comprise one or more of: a comparator, which is arranged to relate sensor values to a series of light intensity patterns, or the like; an energy storage or energy harvesting; a memory storage 10 for storing e.g., measurement values; and an interface 12 for external communication with the external managing device 7. Still other parts of the smart contact lens 2 may be a light manipulation layer that, for example, may be an electro-chromatic layer, and electronics to control the light manipulation layer. Such light manipulation layer may, for instance, be used as a filter layer, e.g., in order to control intensity and/or spectrum of the light transmitted through the smart contact lens 2. In this aspect two approaches may be considered: one comprising active send-detect component and another passive detect-only approach

The external managing device 7 may comprise components such as an interface 8 for communication with the smart contact lens 2 (the device 6 thereof); a time keeping device; a memory storage; a processing unit 9 for various tasks, such as, for instance, processing variations of at least one light intensity value or a series of light intensity values of time to associate to each value a calculated gap-step value. The processing unit 9 of the external managing device 7 may further be arranged to provide such calculated gap-step per time series values to a managing application of e.g., a smart phone.

The managing application may in turn be arranged to receive the gap-step/time series values from the managing device's 7 processing unit and to determine values of cumulative gap-value over time.

When a contact lens is placed on the eye, the lens divides a tear film into two layers, the outermost layer that overlies the lens is called pre-lens tear film (or PrLTF), whereas the layer between the back surface of the contact lens and the cornea is denoted post-lens tear film (PoLTF).

The tear exchange beneath a contact lens, i.e., the fluid circulation between the pre- and the post-lens tear films are commonly referred to as “tear exchange”, “tear turnover”, “tear pumping”, “tear flow”, “tear flushing” or “tear mixing”.

FIGS. 2a and 2b illustrate optical path length difference for light reflected from an upper boundary and a lower boundary of a thin film 11. In optics, a thin film is a layer of material with thickness in the sub-nanometer to micron range. When light strikes an upper surface of the thin film 11, it is either transmitted or reflected. Light that is transmitted through the thin film 11 reaches a bottom surface and may once again be transmitted or reflected. The amount of reflected light may be estimated via the known Fresnel equations. Light reflected from the upper and lower surfaces will eventually interfere, as illustrated in FIG. 2b, where the degree of constructive or destructive interference between the two light waves depends on the difference in their phase.

This difference in phase in turn depends on the thickness of the thin film 11 layer, the refractive index of the thin film 11, and the angle of incidence of the original wave on the thin film 11. Additionally, a phase shift of 180° may be introduced upon reflection (reflected light denoted D) at the upper boundary of the thin film 11, depending on the refractive indices of the materials on either side of the upper boundary. This phase shift occurs if the refractive index of the medium that the light is travelling through is less than the refractive index n of the material it is striking. For instance, in FIG. 2a: if the refractive index n1 of the media through which the incident light is travelling is lower than the refractive index n2 of the media into which the light travels, i.e., n1<n2, then a phase shift θ1 occurs upon reflection (reflected light denoted D). As noted earlier, light that is transmitted through the thin film 11 may also reach the bottom surface B and may once again be transmitted (not illustrated) or reflected (as shown by reflected light indicated by C). The reflected light C then passes the upper surface, with a new phase shift. The pattern of light that results from this interference may typically appear either as light or dark bands or as colorful bands depending on the source of the incident light.

Interference will be constructive (right hand side of FIG. 2b) if the optical path difference caused by the light wave traversing the material with refraction index (n2) is equal to an integer multiple of the wavelength of the light. With phase shift in the material occurring as a combination of the thin film thickness and the refraction index, the phase from the reflection may be destructive (left hand side of FIG. 2b).

With more complex material layering, such as with e.g., anti-reflection coating, oil-film on water, etc., equations describing the respective reflections may typically differ with respect to coefficients for refraction index (and angles associated with respective layers), but same principles still apply.

In case that the incident light is monochromatic (e.g., as emitted from lasers), interference patterns appear as light and dark bands. Light bands correspond to regions at which constructive interference is occurring between the reflected waves and dark bands correspond to destructive interference regions. As the thickness of the film varies from one location to another, the interference may change from constructive to destructive. FIG. 2b illustrates such constructive and destructive phase interaction.

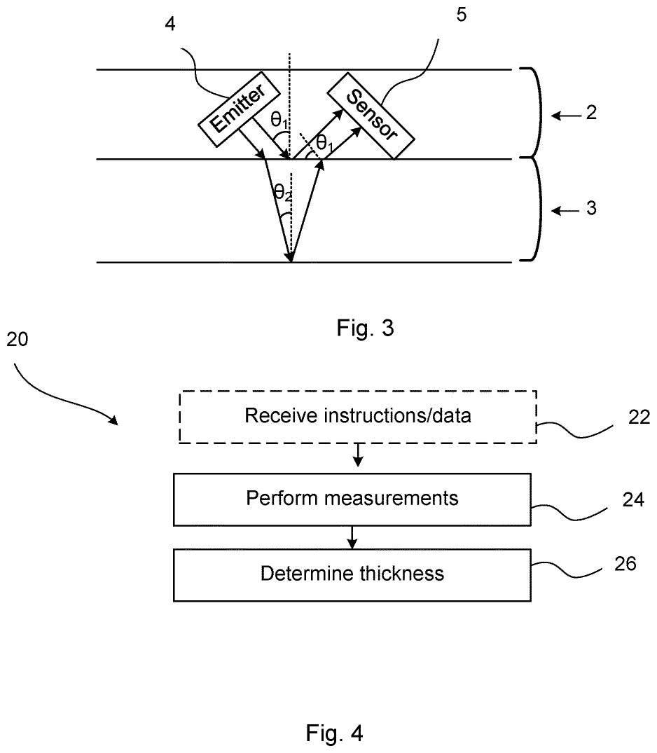

FIG. 3 illustrates a monochromatic interferometer contraption. In the figure, part of the smart contact lens 2 is shown above a part of the thin film, which in this case is the post-lens tear film 3. Following legacy derivation of the theory, equation 1 (Eq. 1) below describes characteristics of the reflected wave in terms of refraction index of the probed material n2 (e.g., the PoLTF), the thin film thickness d and the wavelength of the light source λ, the letter m represents enumeration index.

$$2 {n}_{2} d cos ( {θ}_{2} ) = m λ \qquad ( Eq . 1 )$$

Then given material characteristics such as refraction index of the smart contact lens and tear fluid film, the post lens tear film thickness, and associated angles, can be determined via known relations for thin film interference and Snell's law,

$$2 {n}_{2} d cos ( {θ}_{2} ) = m λ , \qquad ( Eq . 2 )$$

$${n}_{1} sin ( {θ}_{1} ) = {n}_{2} sin ( {θ}_{2} ) , \qquad ( Eq . 3 )$$

respectively, for a scenario of constructive interface (where m=1, 2, 3, 4), i.e., light interference pattern bands. For the scenario with destructive interference and dark bands, then instead m=n−½ and based on Equation 2 and 3, an expression for the post lens tear film thickness (d) in known entities may be established according to:

$$d = \frac{m \cdot λ}{2 \cdot ntear \cdot cos ( θ 2 )} = \frac{m \cdot λ}{2 \cdot ntear \cdot cos ( {sin}^{- 1} ( \frac{nlens}{ntear} \cdot sin ( θ 1 ) ) )} \qquad ( Eq . 4 a )$$

Then, assuming e.g., λ=450 nm, ntear=1.337 [ref], nlens=1.5 [ref] and θ1=45°, a simplified exemplifying expression can be obtained according to:

$$d \approx m \cdot 0.2 [ μm ] \qquad ( Eq . 4 b )$$

Thus, there is two consecutive maximums (such as Δm=1) detected by a sensor 5, and an associated change in lens tear film thickness is obtained by an amount of 0.2 μm.

Based on the above, a method is provided based on a lens-interferometer setup as described earlier, e.g., in relation to FIGS. 1a and 1b. Such method may comprise features such as:
A starting position/starting state where the external managing device 7 communicates with the smart contact lens 2 over a communication interface 8. The communication may comprise information such as, for example, periodicity of light transmitter on/off; periodicity of light sensor readout; associated light intensity-to-sensor array locality/index (intensity value X at sensor position Y; such as [sensor_idx(1:max_len); sensor_intesity(1:max_len)]; light sensor readout intensity-to-parameter values [min . . . max].
One or more operational states where, for instance, a managing application in the external managing device 7 requests data or obtains data from a lens measurement mechanism, wherein the data may comprise any of the above rules and/or attributes. Such measurement data may comprise one or more of:
requesting a light intensity value at a sensor obtained with a periodicity t_p1, at time instance t_i,
determining a first maximum light intensity value Io and associate a first step-value m=m1 to this light intensity value Io,
obtaining, at a later time instance tj>ti, a next light intensity value from a sensor,
determining a second maximum light intensity value Io and associate a second step-value m=m2 to this light intensity value Io, where m2=m1+1,
constructing a value pair of [t_i; d] and store such data values,
subsequently, in operational state with a periodicity of t_p2 (t_p2 less frequent than t_p1), the managing application performs:
   retrieves stored data structure
   [1 time_instance1; d]
   [2 time_instance2; d]
   . . .
   [n time_instance3; d]
   calculates the accumulated measure d_tot as N*d for the number of steps between a first and second selected time entries in the data structure
subsequently, in operational state, providing data to another communication node, such as e.g., the external managing device 7, or an application in a smart phone or in the external managing device 7. Such data or information may comprise information such as time instance, total tear film thickness measure etc.

It is known that a user's eye blink frequency is dependent on type of activity, hour of day, etc., and that the tear film thickness is a function of, among other factors, eye blinking periodicity. Typically, an eye is blinking about 20 times per minute, whereas an eye in front of a screen is blinking with a periodicity, which typically decreases to about 5 times per minute. This is non-desirable from a lens-wearer perspective, e.g., in view of the accumulation of tear film debris and metabolic by-products between the cornea and the smart (soft) contact lens.

To prevent this, the method may comprise steps of selecting the periodicity t_p1 and time instances t_i for light sensor intensity readout and selecting first and second time entries in the data structure.

The steps may be adapted in view of context, e.g., periodicity of sensor readouts may be a function of current eye blinking frequency and/or ongoing activity of the user (e.g., whether the user is in front of a screen or not).

The external managing device 7 (for example a smartwatch) for the device 6 of the smart contact lens 2 may, when receiving the PoLTF data therefrom, also add contextual data such as, for instance, user's activity (e.g., current type of activity) and associated time scheduled/durations.

In a further aspect, the data generated by the device 6 of the smart contact lens 2, may be captured and analyzed in a user device (e.g., a lens case, a smartphone, a smart watch, etc.) and/or forwarded to a central server for further analysis.

Depending on the outcome of such analysis, the central server may send a request to the external managing device 7 about measurements requirements. The external managing device 7 may trigger and monitor measurements based on the request. Examples of such measurements and/or data comprise: time of day and or measurement time window, AND/OR user being at certain locations (at home, in office, outdoors, etc.) AND/OR predicted upcoming user context transitions, such as:
User being in front of display/away from display,
User being indoors/outdoors,
Room temperature/cold or windy outside,
Environment humidity, weather,
Health conditions (user currently fit, or suffering from medical conditions related to an eye illness or a non-eye illness)

The request about measurements requirements may be sent directly to the smart contact lens 2 or be forwarded via the external managing device 7 in order to trigger further measurements, e.g., in a similar way as above.

As an always available kin of optician service, the described embodiments of a method may periodically gather lens data and e.g., suggest exchange of smart contact lens, change of the smart contact lens, etc., and various suggestions deemed appropriate for the specific user, without the user having to pay physical optician/store a visit.

In a further aspect assuming a line array of light sensors (instead of a point sensor) deployed in the smart lens contraption, the method may be evolved from temporal tracking of a first and respective consecutive interference maximum/minimums to an over-array tracking. In that, it will furthermore be understood that in an initial step the line array sensor would provide a series of interference intensity samples per time. Then, the external managing device 7 of the smart contact lens 2 may determine “on-sensor direction” of the interference pattern and determine that one direction corresponds to an increased gap-step value, and the other direction corresponds to an increased gap-step value.

When selecting LED wavelength (frequency) for a typical PoLTF measurement solution, the wavelength may be selected so that “step resolution” of the method corresponds to a typical and/or expected and/or targeted PoLTF gap size. A basic solution assuming any of point or line sensors, may include that measurement solution indicates a deviation from an expected target film thickness value. The sampling should be such that thickness variations should be sampled at a higher frequency than what the to-be-measured film thickness varies with.

Variations in interference pattern intensity over time may furthermore indicate that either the lens or the lens fluid suffer from debris, or similar.

An interference intensity value series being above/below a threshold over time may be a trigger for taking further actions.

Another use of the described method for post-lens tear film gap assessment is for it to operating in conjunction with a smart contact lens capability of measuring eyelid blinking periodicity (the latter known in the arts). This may enable the external managing device 7 to determine if an excessive eyelid blinking is associated with dry eye or some unexpected event such as debris in eye. That is, it would be possible to exclude a dry-eye aspect as the obvious root-cause for excessive blinking.

FIG. 4 is a flowchart of various embodiments of a method according to the herein presented teachings. The method 20 may be used for determining thickness of a post-lens tear film 3 as function of time, and the method 20 may be performed in a device 6 on a smart contact lens 2 placed on an eye 1. It is noted that the device 6 to be placed on the smart contact lens 2 may be added to any type of contact lens, or it may be an integrated part of a smart contact lens 2.

The method 20 comprises performing 24 measurements of light intensity of light transmitted from a light emitter 4 and received by a light detector 5. The light emitter 4 and the light detector 5 are both arranged on the smart contact lens 2.

The method 20 comprises determining 26 the thickness of the post-lens tear film 3 based on the performed measurements, on wavelength of the emitted light and on refraction index of tear fluid between the smart contact lens 2 and the cornea of the eye 1.

In contrast to current in-vivo methods, the herein presented method enables the gathering of contextual aspects of the user wearing the lens; for example, the method 20 enables the user of the smart contact lens 2 to be in any position when gathering information, as opposed to, as in prior art, being located in front of a measurement contraption of an optician or ophthalmologist. In contrast, the present method enables taking into consideration how the user's context such as screen work, etc. impacts the post-lens tear film thickness. Typically, determining quantity and associated flow of and thickness, and composition of tears prior to the contact lens fitting is vital for choosing the appropriate size, material, and wear schedule. The method is advantageous for all types of lenses for eyes, but is particularly beneficial for use of soft lenses, as the exchange of tear beneath the contact lens is considerably lower during the use of this type of lenses compared to use of rigid lenses.

In an embodiment, the determining 26 comprises deriving the thickness of the post-lens tear film 3 as a function of the wavelength of the emitted light and the refraction index of the tear fluid.

In some embodiments, the performing 24 of the measurements comprises measuring the light intensity of repeated transmissions of the light, thereby obtaining a number of measurement results. The determining 26 may then comprise determining interference extrema among the measurement results and determining, for each pair of consecutive extremum comprising two maxima or two minima, a thickness step Δₘ, and determining the thickness of the post-lens tear film to be equal to the thickness step Δₘ multiplied with the number of pairs of consecutive extrema. The extremum comprises an interference maximum or an interference minimum.

In contrast to currently available methods and devices, which require dedicated equipment and measurement arrangements that have a large size and high weight, the present teachings provide a portable device which can be used any time. The devices maybe carried by a user and user context information may be added.

In various embodiments, the light detector 5 is enabled for a light transmission and light reception measuring procedure and has a sampling period shorter than a time constant associated with a post-lens tear film variation. An example of such set-up has been described, e.g., in relation to FIGS. 1a and 1b.

In various different embodiments, method 20 comprises detecting that the determined thickness of the post-lens tear film 3 is below a set threshold thickness and reporting this to an external device 7. In variations of this set of embodiments, the method 20 comprises detecting that a rate of change of the determined thickness is above a set threshold and reporting this to the external device 7. The external device 7 may, for instance, be a user device (e.g., a smart phone, lens case etc.), and the user is then able to perform the method 20 at any time. Such external device, or the smart contact lens 2, may also be able to transmit relevant data to a central server or the like for further analysis. The transmission of data can be done in real-time, or it can be stored in the external device 7 and then sent as a bulk of data. Thus, the rate of change of the thickness should be within certain set limits (thresholds).

Additionally, depending on the outcome of such an analysis, the central server (or other external device) might send a request for additional data and then, for instance, provide parameters for specific contextual fine-tuned measurements, e.g., with an initial indication of a context-dependent tear-film variation, provide managing server/smart lens with an updated measurement schedule.

In an embodiment, the method 20 comprises, prior to the performing 24 measurements, receiving 22, from an external device 7, one or more of: instructions to activate its device 6, periodicity of the light emitter, periodicity of light detector readouts, light detector readout intensity-to-parameter values. This step 22 is illustrated with dashed lines as it is an optional step.

In various embodiments, the method 20 comprises transmitting, to an external device 7, measurement results. In some embodiments, the method 20 comprises receiving, in response to sending the measurement results, one or more of: a request to perform further measurements, specified contextual fine-tuned measurements such as an initial indication of a context-dependent post-lens tear film 3 variation, and receiving an updated measurement schedule or instructions.

In various embodiments, the method 20 comprises using the post-lens tear film thickness information for determining a modified lens-sampling schedule adapted for a specific user.

In various embodiments, the method 20 comprises storing determined information on post-lens tear film thicknesses in a user device. In some embodiments, the user device comprises a smart contact lens 2, a lens case, a smartphone, a smart watch, smart glasses, smart ring, near-eye display device, earbuds and case, headphones, head-mounted display, bracelet, necklace, earrings, piercing, jewelry and smart fabrics clothing.

The present teachings further provide a particular use of the herein described method 20, in any of its embodiments. The use of the smart contact lens 2 for ensuring proper functioning of a post-lens tear film of an eye 1.

A device 6 is also disclosed, the device 20 being configured to perform any of the embodiments, as has been described. The device 6 is configured to determine thickness of a post-lens tear film as function of time, and is arranged on, e.g., as an integrated part of, a smart contact lens 2. The smart contact lens 2 is placed on an eye 1. The device 6 is configured to perform measurements of light intensity of light transmitted from a light emitter 4 and received by a light detector 5, the light emitter 4 and light detector 5 both are part of the device 6 and thus arranged on the smart contact lens 2.

The device 6 is configured to determine the thickness of the post-lens tear film 3 based on the performed measurements, on wavelength of the emitted light and on refraction index of tear fluid between the smart contact lens 2 and the cornea of the eye 1.

In an embodiment, the device 6 is configured to determine by deriving the thickness of the post-lens tear film 3 as a function of the wavelength of the emitted light and the refraction index of the tear fluid.

In various embodiments, the device 6 is configured to measure the light intensity of repeated transmissions of the light, thereby obtaining a number of measurement results. The device 6 is further configured to determine by determining interference extrema among the measurement results; determining, for each pair of consecutive extremum comprising two maxima or two minima, a thickness step Δₘ, and determining the thickness of the post-lens tear film to be equal to the thickness step Δₘ multiplied with the number of pairs of consecutive extrema. The extremum may comprise an interference maximum and/or an interference minimum.

In various embodiments, the light detector 5 is enabled for a light transmission and light reception measuring procedure and has a sampling period shorter than a time constant associated with a post-lens tear film variation.

In various embodiments, the device 6 is configured to detect that the determined thickness of the post-lens tear film 3 is below a set threshold thickness, and to report this to an external device 7. In variations of these embodiments, the device 6 is configured to detect that a rate of change of the determined thickness is above a set threshold, and to report this to the external device 7.

In various embodiments, the device 6 is configured to, prior to the performing measurements, receive, from an external device 7, one or more of: instructions to activate the device 6, periodicity of the light emitter, periodicity of light detector readouts, light detector readout intensity-to-parameter values.

In various embodiments, the device 6 is configured to transmit to an external device 7, measurement results. In variations of these embodiments, the device 6 is configured to receive, in response to sending the measurement results, one or more of: a request to perform further measurements, specified contextual fine-tuned measurements such as an initial indication of a context-dependent post-lens tear film 3 variation, and receiving an updated measurement schedule.

In various embodiments, the device 6 is configured to use the post-lens tear film thickness information for determining a modified lens-sampling schedule adapted for a specific user.

In various embodiments, the device 6 is configured to store determined information on post-lens tear film thicknesses in a user device. The user device may, for instance, comprise a memory of the smart contact lens 2, a lens case, a smartphone, a smart watch, smart glasses, smart ring, near-eye display device, earbuds and case, headphones, head-mounted display, bracelet, necklace, earrings, piercing, jewelry and smart fabrics clothing.

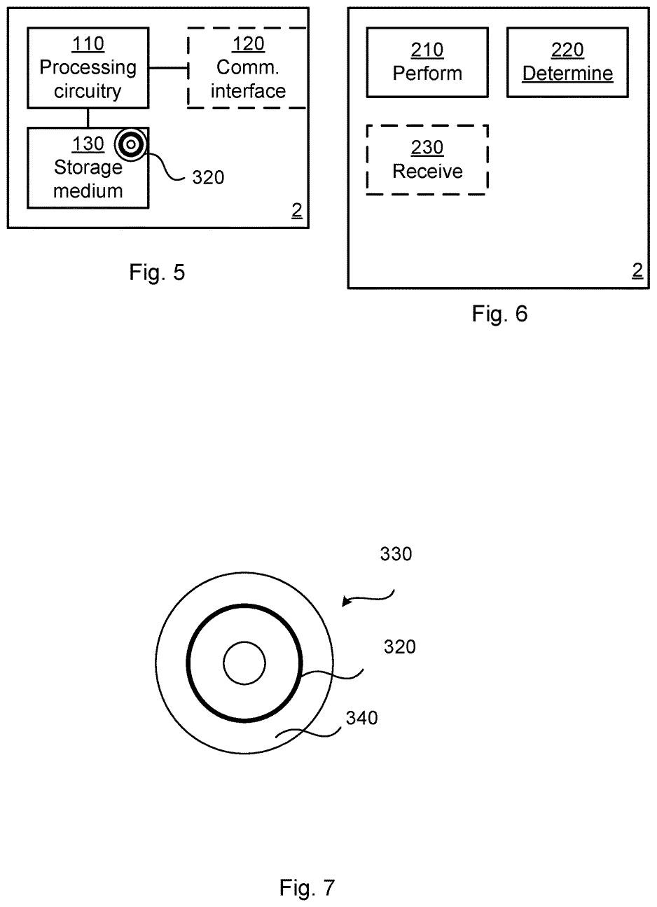

FIG. 5 schematically illustrates, in terms of a number of functional units, the components of a smart contact lens 2 comprising a device 6 (the device 6 may be an integrated part of the smart contact lens 2) as has been described, according to an embodiment. Processing circuitry 110 is provided using any combination of one or more of a suitable central processing unit (CPU), multiprocessor, microcontroller, digital signal processor (DSP), etc., capable of executing software instructions stored in a computer program product 330 (as shown in FIG. 7), e.g., in the form of a storage medium 130. The processing circuitry 110 may further be provided as at least one application specific integrated circuit (ASIC), or field programmable gate array (FPGA).

Particularly, the processing circuitry 110 is configured to cause the smart contact lens 2 to perform a set of operations, or actions, as disclosed above. For example, the storage medium 130 may store the set of operations, and the processing circuitry 110 may be configured to retrieve the set of operations from the storage medium 130 to cause the smart contact lens 2 to perform the set of operations. The set of operations may be provided as a set of executable instructions. The processing circuitry 110 is thereby arranged to execute methods as herein disclosed.

The storage medium 130 may also comprise persistent storage, which, for example, can be any single one or combination of magnetic memory, optical memory, solid state memory or even remotely mounted memory.

The smart contact lens 2 may further comprise a communications interface 120 for communications with other entities, functions, nodes, and devices, over suitable interfaces. As such the communications interface 120 may comprise one or more transmitters and receivers, comprising analogue and digital components.

The processing circuitry 110 controls the general operation of the smart contact lens 2 e.g., by sending data and control signals to the communications interface 120 and the storage medium 130, by receiving data and reports from the communications interface 120, and by retrieving data and instructions from the storage medium 130. Other components, as well as the related functionality, of the smart contact lens 2 are omitted in order not to obscure the concepts presented herein.

FIG. 6 schematically illustrates, in terms of a number of functional modules, the components of a smart contact lens 2 according to an embodiment. The smart contact lens 2 of FIG. 6 comprises a number of functional modules; a perform module 210 configured to perform measurements of light intensity of light transmitted from a light emitter 4 and received by a light detector 5, the light emitter 4 and light detector 5 both being arranged on the smart contact lens 2; and a determine module 220 configured to determine the thickness of the post-lens tear film 3 based on the performed measurements, on wavelength of the emitted light and on refraction index of tear fluid between the smart contact lens 2 and the cornea of the eye 1. The smart contact lens 2 of FIG. 6 may further comprise a number of optional functional modules, such as a receiving module 230 configured to receive, from an external device, one or more of: instructions to activate the device, periodicity of the light emitter, periodicity of light detector readouts, light detector readout intensity-to-parameter values. In general terms, each functional module 210-230 may be implemented in hardware or in software. Preferably, one or more or all functional modules 210-230 may be implemented by the processing circuitry 110, possibly in cooperation with the communications interface 120 and the storage medium 130. The processing circuitry 110 may thus be arranged to from the storage medium 130 fetch instructions as provided by a functional module 210-230 and to execute these instructions, thereby performing any actions of the smart contact lens 2 as disclosed herein.

FIG. 7 shows one example of a computer program product 330 comprising computer readable means 340. On this computer readable means 340, a computer program 320 can be stored, which computer program 320 can cause the processing circuitry 110 and thereto operatively coupled entities and devices, such as the communications interface 120 and the storage medium 130, to execute methods according to embodiments described herein. The computer program 320 and/or computer program product 330 may thus provide means for performing any actions of the smart contact lens 2 as disclosed herein.

In the example of FIG. 7, the computer program product 330 is illustrated as an optical disc, such as a CD (compact disc) or a DVD (digital versatile disc) or a Blu-Ray disc. The computer program product 330 could also be embodied as a memory, such as a random access memory (RAM), a read-only memory (ROM), an erasable programmable read-only memory (EPROM), or an electrically erasable programmable read-only memory (EEPROM) and more particularly as a non-volatile storage medium of a device in an external memory such as a USB (Universal Serial Bus) memory or a Flash memory, such as a compact Flash memory. Thus, while the computer program 320 is here schematically shown as a track on the depicted optical disk, the computer program 320 can be stored in any way which is suitable for the computer program product 330.

The inventive concept has mainly been described above with reference to a few embodiments. However, as is readily appreciated by a person skilled in the art, other embodiments than the ones disclosed above are equally possible within the scope of the inventive concept, as defined by the appended patent claims.

1-29. (canceled)

30. A method for determining thickness of a post-lens tear film as function of time, the method being performed in a device placed on a smart contact lens placed on an eye and comprising:
performing measurements of light intensity of light transmitted from a light emitter and received by a light detector, the light emitter and light detector both being arranged on the smart contact lens; and
determining the thickness of the post-lens tear film based on the performed measurements, a wavelength of the emitted light, and a refraction index of tear fluid between the smart contact lens and the cornea of the eye.

31. The method as claimed in claim 30, wherein the determining comprises deriving, from the performed measurements, the thickness of the post-lens tear film as a function of the wavelength of the emitted light and the refraction index of the tear fluid.

32. The method as claimed in claim 30, wherein performing the measurements comprises:
measuring the light intensity of repeated transmissions of the light, thereby obtaining a number of measurement results;
and wherein the determining comprises:
determining interference extrema among the measurement results; determining, for each pair of consecutive extremum comprising two maxima or two minima, a thickness step Δₘ; and
determining the thickness of the post-lens tear film to be equal to the thickness step Δₘ multiplied with the number of pairs of consecutive extrema.

33. The method as claimed in claim 32, wherein the extremum comprises one of: an interference maximum and an interference minimum.

34. The method as claimed in claim 30, wherein the light detector is enabled for a light transmission and light reception measuring procedure and has a sampling period shorter than a time constant associated with a post-lens tear film variation.

35. The method as claimed in claim 30, comprising detecting that the determined thickness of the post-lens tear film is below a set threshold thickness, and reporting that determination to an external device.

36. The method as claimed in claim 35, comprising detecting that a rate of change of the determined thickness is above a set threshold, and reporting that detection to the external device.

37. The method as claimed in claim 30, comprising, prior to the performing measurements: receiving, from an external device, one or more of: instructions to activate the device, periodicity of the light emitter, periodicity of light detector readouts, light detector readout intensity-to-parameter values.

38. The method as claimed in claim 30, comprising transmitting, to an external device, measurement results.

39. The method as claimed in claim 38, comprising receiving, in response to sending the measurement results, one or more of: a request to perform further measurements, specified contextual fine-tuned measurements such as an initial indication of a context-dependent post-lens tear film variation, and receiving an updated measurement schedule.

40. The method as claimed in claim 30, comprising using the post-lens tear film thickness information for determining a modified lens-sampling schedule adapted for a specific user.

41. The method as claimed in claim 30, comprising storing determined information on post-lens tear film thicknesses in a user device.

42. The method as claimed in claim 41, wherein the user device comprises one of: a lens case, a smartphone, a smart watch, smart glasses, smart ring, near-eye display device, earbuds and case, headphones, head-mounted display, bracelet, necklace, earrings, piercing, jewelry and smart fabrics clothing.

43. A device for determining thickness of a post-lens tear film as function of time, the device being placed on a smart contact lens placed on an eye, the device comprising:
a sensor configured for measurements of light intensity of light transmitted from a light emitter and received by a light detector, the light emitter and light detector both being arranged on the smart contact lens; and
circuitry configured to determine the thickness of the post-lens tear film based on the performed measurements, a wavelength of the emitted light, and a refraction index of tear fluid between the smart contact lens and the cornea of the eye.

44. The device as claimed in claim 43, wherein the circuitry is configured to determine thickness based on deriving, from the performed measurements, the thickness of the post-lens tear film as a function of the wavelength of the emitted light and the refraction index of the tear fluid.

45. The device as claimed in claim 43, wherein the circuitry comprises processing circuitry configured to control the device to:
for performance of the measurements, measure the light intensity of repeated transmissions of the light, thereby obtaining a number of measurement results;
determine interference extrema among the measurement results;
determine, for each pair of consecutive extremum comprising two maxima or two minima, a thickness step Δₘ, and
determine the thickness of the post-lens tear film to be equal to the thickness step Δₘ multiplied with the number of pairs of consecutive extrema.

46. The device as claimed in claim 45, wherein the extremum comprises one of: an interference maximum and an interference minimum.

47. The device as claimed in claim 43, wherein the circuitry is configured to detect that the determined thickness of the post-lens tear film is below a set threshold thickness, and report that determination to an external device via communication interface of the device.

48. The device as claimed in claim 47, wherein the device is comprised in the smart contact lens, wherein the circuitry includes processing circuitry, wherein the device includes a communication interface, and wherein the processing circuitry is configured to use the communication interface to communicate with a smart watch or a user equipment as an external device for controlling or communicating with the device.

49. The device as claimed in claim 48, wherein the processing circuitry is configured to, prior to the device performing the measurements, receive from the external device one or more of: instructions to activate the device, periodicity of the light emitter, periodicity of light detector readouts, or light detector readout intensity-to-parameter values.
